(19) United States
(12) Patent Application Publication
(10) Pub. No.: US 2022/0031239 A1
(43) Pub. Date: Feb. 3, 2022

(54) SYSTEM AND METHOD FOR COLLECTING, ANALYZING AND SHARING BIORHYTHM DATA AMONG USERS

(71) Applicant: Steve CURTIS, Vancouver (CA)

(72) Inventor: Steve CURTIS, Vancouver (CA)

(21) Appl. No.: 17/278,523

(22) PCT Filed: Sep. 21, 2019
(86) PCT No.: PCT/IB2019/058003

§ 371 Date: Mar. 22, 2021

Related U.S. Application Data
(60) Provisional application No. 62/734,490, filed on Sep. 21, 2018.
(60) Provisional application No. 62/734,506, filed on Sep. 21, 2018.
(60) Provisional application No. 62/734,522, filed on Sep. 21, 2018.
(60) Provisional application No. 62/734,608, filed on Sep. 21, 2018.

(51) Int. Cl. A61B 5/00; A61B 5/16; G16H 40/67

(52) U.S. Cl. CPC A61B 5/4857; A61B 5/0022; G16H 40/67; A61B 5/7264; A61B 5/6802; A61B 5/165

(57) ABSTRACT

Disclosed is a system and method to collect, analyze and share biorhythm data among a plurality of users. The method includes the step of collecting biorhythm data of the user through a wearable user device. The method includes the step of receiving the biorhythm data of the users through a computing device communicatively connected with the wearable user device over the communication network and the step of facilitating the users to access the biorhythm data through a synchronization module. The method includes the step of establishing an interaction with the users over the communication network through an artificial intelligence (AI) based agent module. The method includes the step of analyzing and displaying emotional data of the users in real-time through an emotional data displaying module. The method includes the step of modulating biorhythms of the users based on the feedback emitted from the computing device through a feedback module.

### TECHNICAL FIELD

The present invention relates to synchronization of the biorhythm of users, in particular to a system and method for collecting, analyzing and sharing biorhythm data among a plurality of users over a communication network.

### BACKGROUND ART

With the advent of technology, biorhythmic systems and methods have been developed to obtain biorhythmic data of a user and to predict the physical and mental conditions of the user. Typically, biorhythmic data of the humans are calculated by using their personal data and are usually represented by three cycles, intelligence, emotional, and a physical cycle, which oscillate between positive or high periods of activity and negative or low periods of activity.

Usually, communication between users varies in different situations and contexts. For example, in a telephonic conversation, there is an absence of visual while speaking. The user (listener) on the other side of the telephone may not recognize or appreciate subtle changes in the user's (speaker) behavior over an extended period. Also, the user may not realize and understand emotions (depression, stress, happiness, etc.) of another user over the telephone. Therefore, the telephonic conversation may lead to an inefficient method to communicate or interact where both the users do not have an actual understanding of each other's emotions. It is difficult for any existing system to understand the actual emotion attached to the words used by the users. Therefore there is a need for an efficient system that can accurately recognize the emotions of the users during conversation and also determine a good time when the users can talk to each other.

Further, it is important to determine the availability of the user and/or to the user's mental or emotional state to determine whether the time is appropriate for starting any critical or important conversation. Therefore, a user can proactively initiate conversations with other users in a manner that is less likely to be perceived as annoying, invasive, or untimely.

This specification recognizes that there is a need for an efficient and effective method that can collect the biorhythm data and facilitate the users to monitor biorhythm data of the users which can be further shared among the users over a network platform.

Thus, in view of the above, there is a long-felt need in the industry to address the aforementioned deficiencies and inadequacies.

Further limitations and disadvantages of conventional and traditional approaches will become apparent to one of skill in the art through comparison of described systems with some aspects of the present disclosure, as set forth in the remainder of the present application and with reference to the drawings.

### SUMMARY OF INVENTION

A system for collecting, analyzing and sharing biorhythm data among a plurality of users over a communication network is provided substantially, as shown in and/or described in connection with at least one of the figures, as set forth more completely in the claims.

The present invention provides a method to collect, analyze and share biorhythm data among a plurality of users. The method includes the step of collecting biorhythm data of the user through a wearable user device configured to be worn on the user's body, near the body, or placed in the user's body (implantable). The method includes the step of receiving the biorhythm data of the users through a computing device communicatively connected with the wearable user device over the communication network. The method includes the step of facilitating the users to access the biorhythm data through a synchronization module. The method includes the step of establishing an interaction with the users over the communication network through an artificial intelligence (AI) based agent module. The method includes the step of analyzing and displaying emotional data of the users in real-time through an emotional data displaying module. The method includes the step of modulating biorhythms of the users based on the feedback emitted from the computing device through a feedback module.

The synchronization module performs a plurality of steps that initiates with a step of storing the biorhythm data of the users collected by the wearable user device corresponding to the plurality of users through a storage module. The method includes the step of categorizing the biorhythm data stored in the storage module into a plurality of profiles associated with each user through a categorization module. The method includes the step of computing the biorhythm data through a computation module. The method includes the step of communicating the computed data to a network platform through a communication module. The network platform facilitates the users to access the computed data and the profiles of the users connected to the network platform.

The AI-based agent module performs a plurality of steps that initiates with a step of receiving the biorhythm data from the wearable user device and monitors the interactions of a plurality of users and retrieves relevant data for analysis through a tracking module. The tracking module is integrated with one or more messaging platforms and one or more voice platforms of the computing device corresponding to the users to monitor textual interactions and audio interactions of the users. The tracking module processes the relevant data and the retrieved parameters to generate training data. The method includes the step of receiving and processing the training data to determine the emotional state of the user in a plurality of scenarios through a software learning agent module. The method includes the step of initiating the interaction with the user and assist the user based on the learned data received from the software learning agent module through a virtual chat-bot module. The method includes the step of facilitating the user to connect and interact with a plurality of other users through a community module. The community module facilitates the plurality of users to interact with each other and share emotional state and biorhythm data among the other users over the communication network.

The emotional data displaying module performs a plurality of steps that initiates with a step of analyzing the biorhythm data and computing an emotional score of the user to generate one or more insights through an algorithmic module. The emotional score is indicative of the emotional state of the user during the interactions. The method includes the step of graphically representing a plurality of emotional cycles for a specific time duration for the user through a visualization module. The visualization module displays the insights and emotional scores of the users on the computing device associated with the users.

The feedback module performs a plurality of steps that initiates with a step of collecting physiological data of at least one physiological property of the user through a physiological data collection engine. The method includes the step of processing the physiological data into at least one biosignal through a biosignal generating engine. The method includes the step of monitoring and measuring the biosignal for a feedback activation condition through a feedback activation determining engine. The method includes the step of triggering feedback upon satisfying a feedback activation condition through a feedback generating engine. The feedback activation condition triggers feedback when the measured value is more than one or more predetermined threshold values.

In an aspect, the tracking module retrieves a plurality of parameters of the users from the biorhythm data and monitoring data. The plurality of parameters includes the location of the user, biorhythm data of the user, personal and social behavior of the user, and environment, month, day, and time of the interactions.

In an aspect, the plurality of scenarios includes but not limited to contexts, situations, and environments. The software learning agent module is adaptable to continuously learn the contexts, situations, and environments based on the received training data and stores the learned data in a database.

In an aspect, the virtual chat-bot module interacts with the user to assist to improve the emotional state of the user.

In an aspect, the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols which include colors or moving shapes.

Another aspect of the present invention relates to a system for collecting, analyzing and sharing biorhythm data among a plurality of users over a communication network. The system includes a wearable user device and a computing unit. The wearable user device configured to be worn on the user's body, near the body or placed in the user's body (implantable) to collect biorhythm data of the user. The computing unit is communicatively connected with the wearable user device to receive the biorhythm data of the users over a communication network. The computing unit includes a processor, and a memory communicatively coupled to the processor. The memory includes a synchronization module, an artificial intelligence (AI) based agent module, an emotional data displaying module, and a feedback module.

The synchronization module facilitates the users to access the biorhythm data over a network platform. The artificial intelligence (AI) based agent module establishes an interaction with the users over the communication network. The emotional data displaying module analyzes and displays emotional data of the users in real-time. The feedback module configured with the wearable user device to modulate biorhythms of the users based on the feedback emitted from the computing device.

The synchronization module includes a storage module, a categorization module, a computation module, and a communication module. The storage module stores the biorhythm data of the users collected by the wearable user device corresponding to the plurality of users. The categorization module categorizes the biorhythm data stored in the storage module into a plurality of profiles associated with each user. The computation module computes the biorhythm data. The communication module communicates the computed data to a network platform. The network platform facilitates the users to access the computed data and the profiles of the users connected to the network platform.

The AI-based agent module includes a tracking module, a software learning agent module, a virtual chat-bot module, and a community module. The tracking module receives the biorhythm data from the wearable user device and monitors the interactions of a plurality of users and retrieves relevant data for analysis. The tracking module is integrated with one or more messaging platforms and one or more voice platforms of the computing device corresponding to the users to monitor textual interactions and audio interactions of the users. The tracking module processes the relevant data and the retrieved parameters to generate training data. The software learning agent module receives and processes the training data to determine the emotional state of the user in a plurality of scenarios. The virtual chat-bot module initiates the interaction with the user and assists the user based on the learned data received from the software learning agent module. The community module facilitates the user to connect and interact with a plurality of other users. The community module facilitates the plurality of users to interact with each other and share emotional state and biorhythm data among the other users over the communication network.

The emotional data displaying module includes an algorithmic module and a visualization module. The algorithmic module analyzes the biorhythm data and computes an emotional score of the user to generate one or more insights. The emotional score is indicative of the emotional state of the user during the interactions. The visualization module graphically represents a plurality of emotional cycles for a specific time duration for the user. The visualization module displays the insights and emotional scores of the users on the computing device associated with the users.

The feedback module includes a physiological data collection engine, a biosignal generating engine, a feedback activation determining engine, and a feedback generating engine. The physiological data collection engine collects physiological data of at least one physiological property of the user. The biosignal generating engine processes the physiological data into at least one biosignal. The feedback activation determining engine monitors and measures the biosignal for a feedback activation condition. The feedback generating engine triggers feedback upon satisfying a feedback activation condition. The feedback activation condition triggers feedback when the measured value is more than one or more predetermined threshold values.

In an aspect, the present system enables the user to login to a native application installed within the computing device of the user. The native application displays names of the users corresponding to the profiles associated with each user. Further, the users are enabled to access the profile and biorhythm data through the synchronization module.

Accordingly, one advantage of the present invention is that it provides multi-syncing for synchronization of a plurality of user accounts for sharing biorhythm data of each other for accurate and efficient communication.

Accordingly, one advantage of the present invention is that it controls (increase or decrease) an involuntary or unconscious physiological process by self-regulating and exercising control over physiological variables.

Accordingly, one advantage of the present invention is that it provides a social platform to the users where they share their emotional data and allow other users to visualize the same to improve and work on their emotional state.

Accordingly, one advantage of the present invention is that it improves communication amongst the users based on the biorhythmic data.

Accordingly, one advantage of the present invention is that the computing device displays related sync results to offer visual, auditory, or haptic/tactile feedback that progressively synchronizes various behaviors among users.

Accordingly, one advantage of the present invention is that it efficiently moves the users with a negative emotional state to the users with a positive emotional state to yield a more positive conversational experience between the users.

Other features of embodiments of the present invention will be apparent from accompanying drawings and from the detailed description that follows.

Yet other objects and advantages of the present invention will become readily apparent to those skilled in the art following the detailed description, wherein the preferred embodiments of the invention are shown and described, simply by way of illustration of the best mode contemplated herein for carrying out the invention. As we realized, the invention is capable of other and different embodiments, and its several details are capable of modifications in various obvious respects, all without departing from the invention. Accordingly, the drawings and description thereof are to be regarded as illustrative in nature, and not as restrictive.

### BRIEF DESCRIPTION OF DRAWINGS

In the figures, similar components and/or features may have the same reference label. Further, various components of the same type may be distinguished by following the reference label with a second label that distinguishes among the similar components. If only the first reference label is used in the specification, the description applies to any one of the similar components having the same first reference label irrespective of the second reference label.

FIG. 1 illustrates a block diagram of the present system for collecting, analyzing and sharing biorhythm data among a plurality of users over a communication network, in accordance with one embodiment of the present invention.

FIG. 2 illustrates a network implementation of the present system, in accordance with one embodiment of the present invention.

FIG. 3 illustrates a block diagram of the various modules within a memory of a computing device, in accordance with another embodiment of the present invention.

FIG. 4 illustrates a flowchart of the method for collecting, analyzing and sharing biorhythm data among a plurality of users over a communication network, in accordance with an alternative embodiment of the present invention.

FIG. 5 illustrates a flowchart of the plurality of steps performed by a synchronization module, in accordance with an alternative embodiment of the present invention.

FIG. 6 illustrates a flowchart of the plurality of steps performed by an artificial intelligence (AI) based agent module, in accordance with an alternative embodiment of the present invention.

FIG. 7 illustrates a flowchart of the plurality of steps performed by an emotional data displaying module, in accordance with an alternative embodiment of the present invention.

FIG. 8 illustrates a flowchart of the plurality of steps performed by a feedback module, in accordance with an alternative embodiment of the present invention.

### DESCRIPTION OF EMBODIMENTS

The present disclosure is best understood with reference to the detailed figures and description set forth herein. Various embodiments have been discussed with reference to the figures. However, those skilled in the art will readily appreciate that the detailed descriptions provided herein with respect to the figures are merely for explanatory purposes, as the methods and systems may extend beyond the described embodiments. For instance, the teachings presented and the needs of a particular application may yield multiple alternative and suitable approaches to implement the functionality of any detail described herein. Therefore, any approach may extend beyond certain implementation choices in the following embodiments.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

Methods of the present invention may be implemented by performing or completing manually, automatically, or a combination thereof, selected steps or tasks. The term “method” refers to manners, means, techniques and procedures for accomplishing a given task including, but not limited to, those manners, means, techniques, and procedures either known to, or readily developed from known manners, means, techniques and procedures by practitioners of the art to which the invention belongs. The descriptions, examples, methods, and materials presented in the claims and the specification are not to be construed as limiting but rather as illustrative only. Those skilled in the art will envision many other possible variations within the scope of the technology described herein.

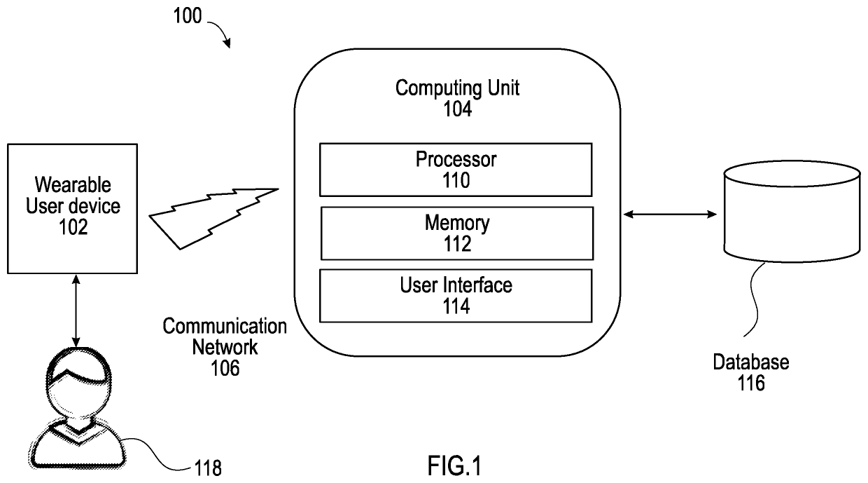

FIG. 1 illustrates a block diagram of the present system 100 for collecting, analyzing and sharing biorhythm data among a plurality of users over a communication network, in accordance with one embodiment of the present invention. In an embodiment, the system 100 may share other non-biorhythmic data such as personal data, and emotional data of the users or any other scores computed by the various modules of the present system 100. The system 100 includes a wearable user device 102, and a computing device 104. The wearable user device 102 is configured to be worn on the user's body, near the body, or placed in the user's body (implantable) to collect biorhythm data of the user 118. Examples of the wearable user device 102 include but not limited to the implantable, wireless sensor device, smartwatch, smart jewelry, fitness tracker, smart cloth, etc. In an embodiment, the wearable user device 102 includes various sensors to detect one or more parameters pertaining to the emotions of the user 118. In an embodiment, the wearable user device 102 may include a flexible body that can be secured around the user's body to collect the biorhythm data. In an embodiment, and the wearable user device 102 may including a securing mechanism to secure the wearable user device 102 may in a closed loop around a wrist of the user 118. Further, the wearable user device 102 may be any wearable such as an on-body sticker or 3d-printed device that is directly printed on the skin, or a device that placed on the body with an adhesive. The wearable user device 102 may utilize various wired or wireless communication protocols to establish communication with the computing unit 104.

The computing device 104 is communicatively connected with the wearable user device 102 to receive the biorhythm data of the users over a communication network 106.

Communication network 106 may be a wired or a wireless network, and the examples may include but are not limited to the Internet, Wireless Local Area Network (WLAN), Wi-Fi, Long Term Evolution (LTE), Worldwide Interoperability for Microwave Access (WiMAX), General Packet Radio Service (GPRS), Bluetooth (BT) communication protocols, Transmission Control Protocol and Internet Protocol (TCP/IP), User Datagram Protocol (UDP), Hypertext Transfer Protocol (HTTP), File Transfer Protocol (FTP), ZigBee, EDGE, infrared (IR), Z-Wave, Thread, 5G, USB, serial, RS232, NFC, RFID, WAN, and/or IEEE 802.11, 802.16, 2G, 3G, 4G cellular communication protocols

Examples of the computing device 104 include but not limited to a laptop, a desktop, a smartphone, a smart device, a smartwatch, a phablet, a body implant, smart glass, and a tablet. The computing device 104 includes a processor 110, a memory 112 communicatively coupled to the processor 110, and a user interface 114. The computing device 104 is communicatively coupled with a database 114. The database 116 receives, stores, and processes the emotional data and referral data which can be used for further analysis and prediction so that the present system can learn and improve the analysis by using the historical emotional data. Although the present subject matter is explained considering that the present system 100 is implemented on a cloud device, it may be understood that the present system 100 may also be implemented in a variety of computing systems, such as an Amazon elastic compute cloud (Amazon EC2), a network server, and the like. The data collected from the user is constantly being monitored and sent to the server (when convenient and connected), where it is stored, analyzed, and modeled. New AI models are generated on the server and then downloaded to the computing devices at various intervals.

Processor 110 may include at least one data processor for executing program components for executing user- or system-generated requests. A user may include a person, a person using a device such as those included in this invention, or such a device itself. Processor 110 may include specialized processing units such as integrated system (bus) controllers, memory management control units, floating-point units, graphics processing units, digital signal processing units, etc.

Processor 110 may include a microprocessor, such as AMD® ATHLON® microprocessor, DURON® microprocessor OR OPTERON® microprocessor, ARM's application, embedded or secure processors, IBM® POWERPC®, INTEL'S CORE® processor, ITANIUM® processor, XEON® processor, CELERON® processor or other line of processors, etc. Processor 110 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor 110 may be disposed of in communication with one or more input/output (I/O) devices via an I/O interface. I/O interface may employ communication protocols/methods such as, without limitation, audio, analog, digital, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), etc.

Memory 112, which may be a non-volatile memory or a volatile memory. Examples of non-volatile memory may include, but are not limited to flash memory, a Read Only Memory (ROM), a Programmable ROM (PROM), Erasable PROM (EPROM), and Electrically EPROM (EEPROM) memory. Examples of volatile memory may include but are not limited Dynamic Random Access Memory (DRAM), and Static Random-Access memory (SRAM).

The user interface 114 may present the collected data, analyzed data and shared biorhythm data as per the request of an administrator or users of the present system. In an embodiment, the user interface (UI or GUI) 114 is a convenient interface for accessing the platform and viewing the products or services. The biorhythmic data includes but not limited to heart rate, heart rate variability, electrodermal activity (EDA)/Galvanic skin response (GSR), breathing rate, 3D accelerometer data, and gyroscope data, body temperature, a pulse rate, respiratory rate, electrocardiography (ECG), skin temperature, brain waves such as electroencephalography (EEG), electrooculography (EOG), blood pressure, hydration level among others. The biorhythmic data can be processed to generate the signals based on mathematical description or algorithms. The algorithms may be introduced via software. There is potential that data is processed on the wearable user device end. Data may also be stored there temporarily before acted upon.

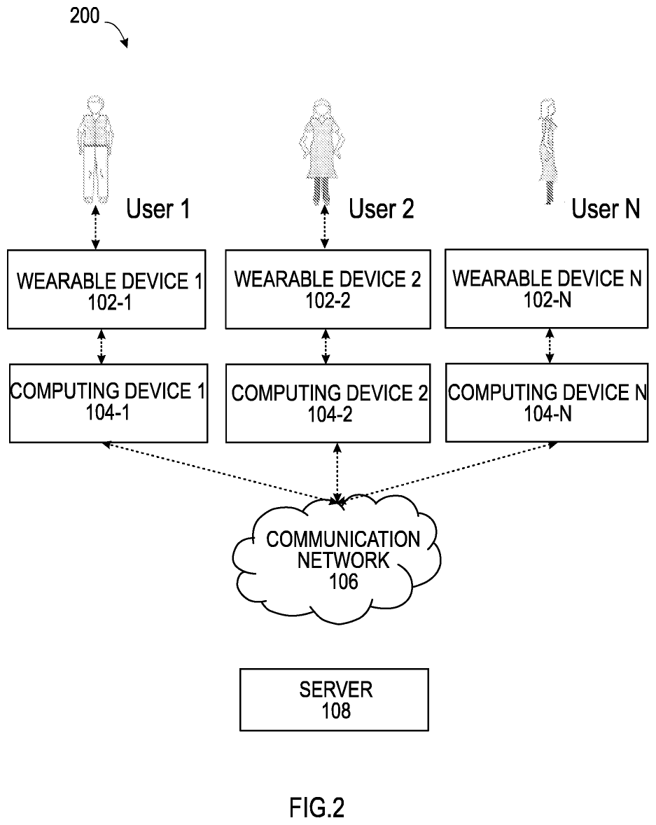

FIG. 2 illustrates a network implementation 200 of the present system, in accordance with one embodiment of the present invention. FIG. 2 is explained in conjunction with FIG. 1. The computing devices 104-1, 104-2, and 104-N are communicatively connected with the wearable user devices 102-1, 102-2, and 102-N to receive the biorhythm data of the users over the communication network 106. A server 108 stores and processes the monitored interaction data, determine emotional data and modulated biorhythms data. The computing device 104 or wearable user device 102 may initiate a sound notification (any type of sound). Based on the user's current emotional state score, different sounds should be issued by one or more of the wearable user devices 102 to inform the users to do one of several different behaviors. It may be appreciated that behavior may not be limited to one behavior, and sound could signal a plurality (multiple) of actions. The behavior associated with the sound should help the user change their behavior to move closer to the user's desired/preset emotional state, or move towards changing a more specific biorhythm.

In an aspect, the network architecture of the wearable user device 102 and the computing device 104 can include one or more Internet of Things (IoT) devices. In a typical network architecture of the present disclosure can include a plurality of network devices such as transmitter, receivers, and/or transceivers that may include one or more IoT devices.

In an aspect, the wearable user device, 102 can directly interact with the cloud and/or cloud servers and IoT devices. The data and/or information collected can be directly stored in the cloud server without taking any space on the user mobile and/or portable computing device. The mobile and/or portable computing device can directly interact with a server and receive information for feedback activation to trigger and deliver the feedback. Examples of the feedback include but not limited to auditory feedback, haptic feedback, tactile feedback, vibration feedback, or visual feedback from a primary wearable device, a secondary wearable device, a separate computing device (i.e. mobile), or IoT device (which may or may not be a computing device). In an embodiment, a primary wearable device, a secondary wearable device, another/separate computing device, and/or IoT device may provide various feedbacks such as visual feedback, haptic or tactile or vibration feedback. The visual feedback may be in the form of a pulse or flash sequence of a specific wavelength light or across a variety of visible wavelengths (multiple colors). The light or lights may dim or brighten or may change color, may turn on or off, or change flashing sequence or any combination of these to indicate a change has occurred. Haptic/tactile/ or vibration feedback such that vibration can be physically detected on the skin or the vibration heard from a 15-meter range—in the same room. This may pulse, change vibration frequency/speeds or change amplitude (to increase or decrease the strength of the vibration).

As used herein, the IoT devices can be a device that includes sensing and/or control functionality as well as a WiFi™ transceiver radio or interface, a Bluetooth™ transceiver radio or interface, a Zigbee™ transceiver radio or interface, an Ultra-Wideband (UWB) transceiver radio or interface, a WiFi-Direct transceiver radio or interface, a Bluetooth™ Low Energy (BLE) transceiver radio or interface, and/or any other wireless network transceiver radio or interface that allows the IoT device to communicate with a wide area network and with one or more other devices. In some embodiments, an IoT device does not include a cellular network transceiver radio or interface, and thus may not be configured to directly communicate with a cellular network. In some embodiments, an IoT device may include a cellular transceiver radio and may be configured to communicate with a cellular network using the cellular network transceiver radio.

A user may communicate with the network devices using an access device that may include any human-to-machine interface with network connection capability that allows access to a network. For example, the access device may include a stand-alone interface (e.g., a cellular telephone, a smartphone, a home computer, a laptop computer, a tablet, a personal digital assistant (PDA), a computing device, a wearable device such as a smartwatch, a wall panel, a keypad, or the like), an interface that is built into an appliance or other device e.g., a television, a refrigerator, a security system, a game console, a browser, or the like), a speech or gesture interface (e.g., a Kinect™ sensor, a Wiimote™, or the like), an IoT device interface (e.g., an Internet-enabled devices such as a wall switch, a control interface, or other suitable interface), or the like. In some embodiments, the access device may include a cellular or other broadband network transceiver radio or interface and may be configured to communicate with a cellular or other broadband network using the cellular or broadband network transceiver radio. In some embodiments, the access device may not include a cellular network transceiver radio or interface.

In an embodiment, the users may be provided with an input/display screen which is configured to display information to the user about the current status of the system. The input/display screen may take input from an input apparatus, in the current example buttons. The input/display screen may also be configured as a touch screen or may accept input for determining vitals or bio-signals through touch or haptic based input system. The input buttons and/or screen are configured to allow a user to respond to input prompt from the system regarding needed user input.

The information which may be displayed on the screen to the user may be, for instance, the number of treatments provided, bio-signals values, vitals, the battery charge level, and volume level. The input/display screen may take information from a processor which may also be used as the waveform generator or maybe a separate processor. The processor provides available information for display to the user allowing the user to initiate menu selections. The input/display screen may be a liquid crystal display to minimize power drain on the battery. The input/display screen and the input buttons may be illuminated to provide a user with the capability to operate the system in low light levels. Information can be obtained from a user through the use of the input/display screen.

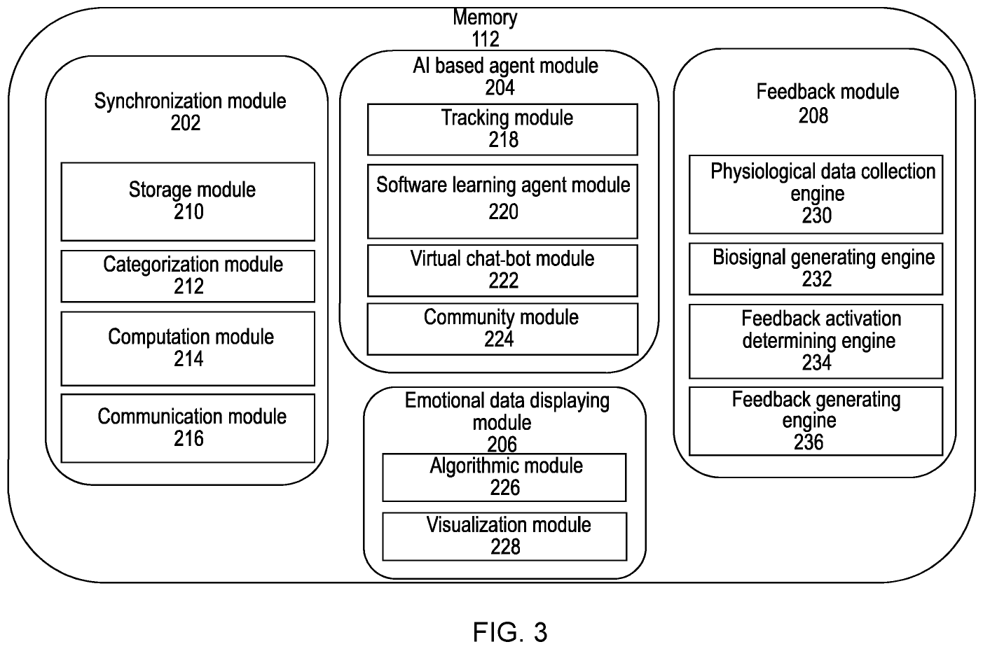

FIG. 3 illustrates a block diagram of the various modules within a memory 112 of a computing device 104, in accordance with another embodiment of the present invention. FIG. 3 is explained in conjunction with FIG. 1. The memory 110 includes synchronization module 202, an artificial intelligence (AI) based agent module 204, an emotional data displaying module 206, and a feedback module 208.

The synchronization module 202 facilitates the users to access the biorhythm data over a network platform. The artificial intelligence (AI) based agent module 204 establishes an interaction with the users over the communication network. The emotional data displaying module 206 analyzes and displays emotional data of the users in real-time. The feedback module 208 configured with the wearable user device to modulate biorhythms of the users based on the feedback emitted from the computing device.

The synchronization module 202 includes a storage module 210, a categorization module 212, a computation module 214, and a communication module 216. The storage module 210 stores the biorhythm data of the users collected by the wearable user device corresponding to the plurality of users. The categorization module 212 categorizes the biorhythm data stored in the storage module into a plurality of profiles associated with each user. The computation module 214 computes the biorhythm data. The computation module 214 synthesizes insights based on the various combinations and computations of the biorhythm data. For example, a low pulse rate combined with low breathing combined with little to no movement may indicate a user is sleeping. The communication module 216 communicates the computed data to a network platform. The network platform facilitates the users to access the computed data and the profiles of the users connected to the network platform. However, the synchronization module 202 may enable the users to secure their data related to biorhythm, emotion, personal information, etc. to safeguard their privacy. Thus the users may have complete control of their data. In an embodiment, the network platform may include a native application or a social media platform which can be utilized to achieve the various objectives of the present system.

In an embodiment, the synchronization module 202 allows the user to access the emotion data of the other users over the network platform. The network platform of the synchronization module 202 may utilize an initiation and acceptance protocols to enables the user to accept/decline the friend request and allow/disallow the other users to access his/her emotional data. Alternatively, the users may turn on a setting that is (bidirectional or unidirectional) to allow both the users to receive unlimited access to the one or each other's data. Regardless of the protocol, and directionality of the sync, the end benefit is that the other person's psychological state or emotional state score should be visualized with options to view past periods of time. Most importantly, assuming real-time data is streaming from each other's devices to their secondary devices (mobile phones), the users should be able to view each other's real-time emotional scores. These emotional scores can be divided into zones that can be linearly divided or along zones in a 2-axis array, or in zones based on the n-dimensional matrix. Overall, the zones follow some clear gradient that is communicated to users in various places in the product. The syncing states between two parties also allow evaluations to be made and insights to be derived between the 2 or more synced accounts.

In an additional embodiment, the synchronization module 202 may use a multi-syncing module. The multi-syncing module enables more than two user accounts to sync up. The use of location-based services facilitates easy recognition when multi-syncing can occur. If multiple devices are detected on a software application associated with the synchronization module 202 or if the GPS services detect that computing units are within a short distance of each other, then those users—who have already acknowledged each other as friends on the community module—will appear most prominent on the list.

The multi-syncing module provides advanced insights and shows many groups statistics. The notifications in the multi-syncing module may include changes in groups results. In an embodiment, the sync factor can be turned off at any given time by anyone. In the multi-syncing module, if one user turns off their sync feature, the feature will persist for other group members. The secondary computing units that display related sync results may offer visual, auditory, or haptic/tactile feedback that progressively synchronizes various behaviors such as breathing rate and aspect of the breathing cycle (whether both people are at the peak of inhalation or trough of exhalation). Further, the sync feature encompasses applies to any combinations of biorhythms including brain waves such as EEG.

In an embodiment, the software application identifies the target points on markers, or users can mutually or individually select goals/targets points for biorhythm measurements. Once these targets are identified, the feedback of various types will then work change behavior and biorhythms to move them closer to this target point. The target can be static or dynamic. The objective of the syncing is to move the emotional states of the two or more users closer together, but only in a positive direction. Moving one user who is in a negative emotional state to closer alignment with a person in a positive emotional state will yield a more positive conversational experience between the two users.

In an embodiment, the synchronization module 202 comprises a recording module to record the conversation. The recording module acts as a virtual button over an interface that allows the user to turn ON/OFF the recording. Audio is then recorded through the microphone of a secondary computing unit if there is one or a similar tool available. The synchronization module 202 comprises a language processing module that applies to the recorded audio files to transform the dialogue audio waves into the transcribed language. The transcribed language is further processed based on sentiment and content and matched temporally with biorhythms of the speaker's emotional scores.

In an embodiment, the computing device may provide a coaching mechanism that acts as a coach after synching two or more biorhythm data of the users. The coaching mechanism can be selected from a secondary user, a computerized smart agent such as a bot or a combination of both and acts as a therapist, counselor, physician, facilitator, or mediator. The coach can perform various actions such as view profiles of the users, personalized data that includes but not limited to demographic, psychographic, computed statistics or scores, other data shared by users, or perhaps externally pulled data on users from outside sources about any of the users. The coach may be enabled to control the synchronization among the users and can prevent them from messaging with each other.

The AI-based agent module 204 includes a tracking module 218, a software learning agent module 220, a virtual chat-bot module 222, and a community module 224. The tracking module 218 receives the biorhythm data from the wearable user device and monitors the interactions of a plurality of users and retrieves relevant data for analysis. The tracking module 218 is integrated with one or more messaging platforms and one or more voice platforms of the computing device corresponding to the users to monitor textual interactions and audio interactions of the users. The tracking module 218 processes the relevant data and the retrieved parameters to generate training data. In an embodiment, the tracking module 218 retrieves a plurality of parameters of the users from the biorhythm data and monitored data. The plurality of parameters includes the location of the user, biorhythm data of the user, personal and social behavior of the user, and environment, month, day, and time of the interactions. In an embodiment, the plurality of scenarios includes but not limited to contexts, situations, and environments.

The software learning agent module 220 receives and processes the training data to determine the emotional state of the user in a plurality of scenarios. In an embodiment, the training data can be combined or deconstructed or converted in various ways to aid modeling. The training data can be utilized to train the various algorithms used to achieve the objective of the present system. The training data includes input data and the corresponding expected output. Based on the training data, the algorithm can learn how to apply various mechanisms such as neural networks, to learn, produce, and predict the emotional state of the user in the plurality of scenarios, so that it can accurately determine the emotional state when later presented with new input data.

The software learning agent module 220 is adaptable to continuously learn the contexts, situations, and environments based on the received training data and stores the learned data in a database. The virtual chat-bot module 222 initiates the interaction with the user and assist the user based on the learned data received from the software learning agent module. In an embodiment, the virtual chat-bot module 222 interacts with the user to assist to improve the emotional state of the user.

The community module 224 facilitates the user to connect and interact with a plurality of other users. The community module 224 facilitates the plurality of users to interact with each other and share emotional state and biorhythm data among the other users over the communication network. The community module 224 enables the user to view a list of existing friends and further enables the user to search for other users via a text-based name search. The users can also send friend requests to other users. The other users receive a notification on receiving the friend request from the users. The users can accept or decline the friend request. The community module 224 further allows both the users to access the general statistics related to the emotional state of each other. Additionally, the user can interact with each other through a messaging module integrated within the community module 224. The user is presented with various options to communicate with the profiles of the users that include but not limited to chat, phone call, send a friend request, add to contact, sync module, multi-sync, send profile information and others. The native application enables the user to search other users via specifying personal details in the search text box module.

The emotional data displaying module 206 includes an algorithmic module 226, and a visualization module 228. The algorithmic module 226 analyzes the biorhythm data and computes an emotional score of the user to generate one or more insights. The emotional score is indicative of the emotional state of the user during the interactions. The visualization module 228 graphically represents a plurality of emotional cycles for a specific time duration for the user. The visualization module 228 displays the insights and emotional scores of the users on the computing device associated with the users. In an embodiment, the visualization module 228 displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols which include colors or moving shapes.

The feedback module 208 includes a physiological data collection engine 230, a biosignal generating engine 232, a feedback activation determining engine 234, and feedback generating engine 236. The physiological data collection engine 230 collects physiological data of at least one physiological property of the user. The biosignal generating engine 232 processes the physiological data into at least one biosignal. The feedback activation determining engine monitors and measures the biosignal for a feedback activation condition. The feedback generating engine 236 triggers feedback upon satisfying a feedback activation condition. The feedback activation condition triggers feedback when the measured value is more than one or more predetermined threshold values.

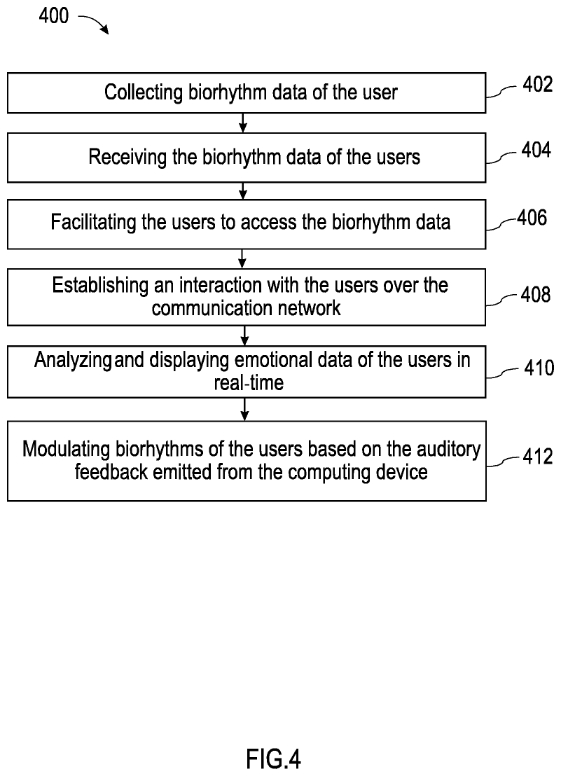

FIG. 4 illustrates a flowchart 400 of the method for collecting, analyzing and sharing biorhythm data among a plurality of users over a communication network, in accordance with an alternative embodiment of the present invention. The method includes step 402 of collecting biorhythm data of the user through a wearable user configured to be worn on the user's body, near the body, or placed in the user's body (implantable). The method includes the step 404 of receiving the biorhythm data of the users through a computing device communicatively connected with the wearable user device over the communication network. The method includes the step 406 of facilitating the users to access the biorhythm data through a synchronization module. The method includes the step 408 of establishing an interaction with the users over the communication network through an artificial intelligence (AI) based agent module. The method includes step 410 of analyzing and displaying emotional data of the users in real-time through an emotional data displaying module. The method includes the step 412 of modulating biorhythms of the users based on the feedback emitted from the computing device through a feedback module.

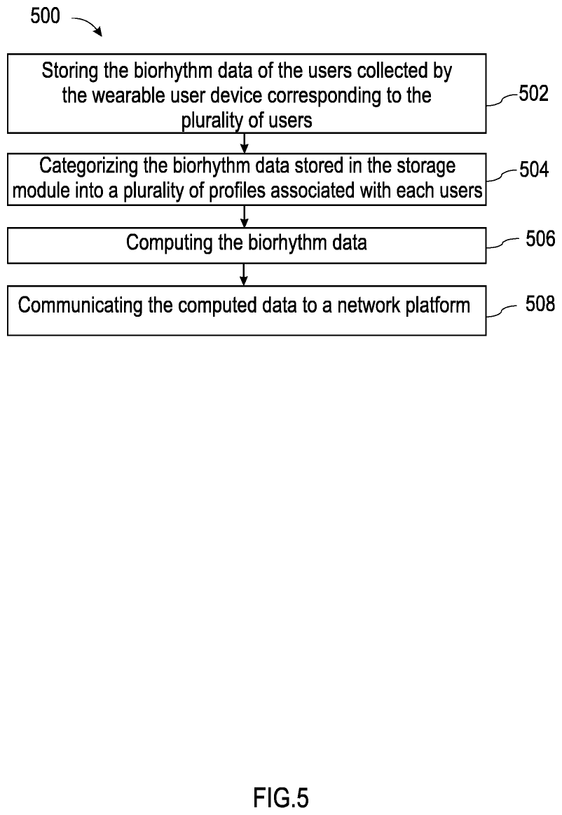

FIG. 5 illustrates a flowchart 500 of the plurality of steps performed by a synchronization module, in accordance with an alternative embodiment of the present invention. The synchronization module performs a plurality of steps that initiates with a step 502 of storing the biorhythm data of the users collected by the wearable user device corresponding to the plurality of users through a storage module. The method includes the step 504 of categorizing the biorhythm data stored in the storage module into a plurality of profiles associated with each user through a categorization module. The method includes the step 506 of computing the biorhythm data through a computation module. The method includes the step 508 of communicating the computed data to a network platform through a communication module. The network platform facilitates the users to access the computed data and the profiles of the users connected to the network platform.

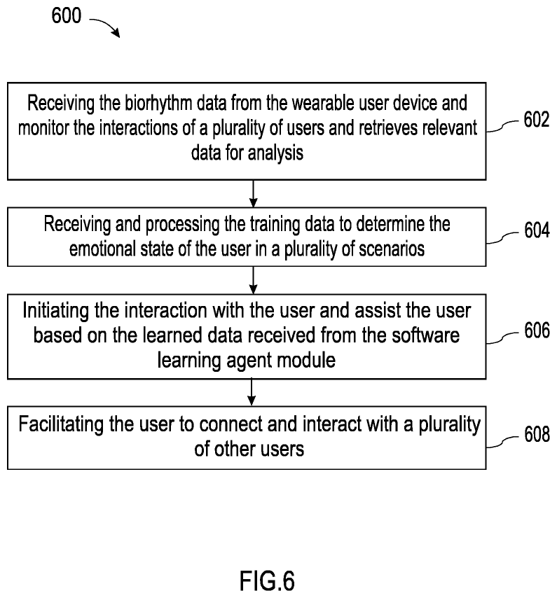

FIG. 6 illustrates a flowchart 600 of the plurality of steps performed by an artificial intelligence (AI) based agent module, in accordance with an alternative embodiment of the present invention. The AI-based agent module performs a plurality of steps that initiates with a step 602 of receiving the biorhythm data from the wearable user device and monitor the interactions of a plurality of users and retrieves relevant data for analysis through a tracking module. The tracking module is integrated with one or more messaging platforms and one or more voice platforms of the computing device corresponding to the users to monitor textual interactions and audio interactions of the users. The tracking module processes the relevant data and the retrieved parameters to generate training data. In an embodiment, the tracking module retrieves a plurality of parameters of the users from the biorhythm data and monitoring data. The plurality of parameters includes the location of the user, biorhythm data of the user, personal and social behavior of the user, and environment, month, day, and time of the interactions. In an embodiment, the plurality of scenarios includes but not limited to contexts, situations, and environments. The software learning agent module is adaptable to continuously learn the contexts, situations, and environments based on the received training data and stores the learned data in a database.

The method includes step 604 of receiving and processing the training data to determine the emotional state of the user in a plurality of scenarios through a software learning agent module. The method includes step 606 of initiating the interaction with the user and assist the user based on the learned data received from the software learning agent module through a virtual chat-bot module. In an embodiment, the virtual chat-bot module interacts with the user to assist to improve the emotional state of the user. The method includes the step 608 of facilitating the user to connect and interact with a plurality of other users through a community module. The community module facilitates the plurality of users to interact with each other and share emotional state and biorhythm data among the other users over the communication network.

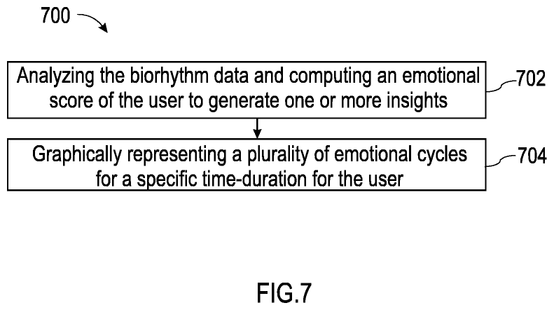

FIG. 7 illustrates a flowchart 700 of the plurality of steps performed by an emotional data displaying module, in accordance with an alternative embodiment of the present invention. The emotional data displaying module performs a plurality of steps that initiates with a step 702 of analyzing the biorhythm data and computing an emotional score of the user to generate one or more insights through an algorithmic module. The emotional score is indicative of the emotional state of the user during the interactions. The method includes the step 704 of graphically representing a plurality of emotional cycles for a specific time duration for the user through a visualization module. The visualization module displays the insights and emotional scores of the users on the computing device associated with the users. In an embodiment, the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols which include colors or moving shapes.

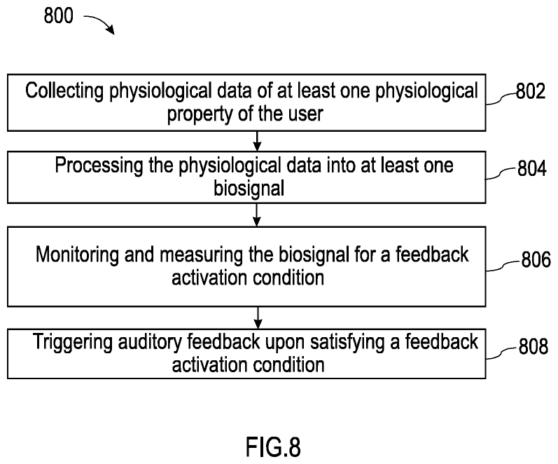

FIG. 8 illustrates a flowchart 800 of the plurality of steps performed by a feedback module, in accordance with an alternative embodiment of the present invention. The feedback module performs a plurality of steps that initiates with a step 802 of collecting physiological data of at least one physiological property of the user through a physiological data collection engine. The method includes the step 804 of processing the physiological data into at least one biosignal through a biosignal generating engine. The method includes the step 806 of monitoring and measuring the biosignal for a feedback activation condition through a feedback activation determining engine. The method includes step 808 of triggering feedback upon satisfying a feedback activation condition through feedback generating engine. The feedback activation condition triggers feedback when the measured value is more than one or more predetermined threshold values.

Thus the present system and method provide a network platform that utilizes the synchronization module to allow the users to view biorhythm data of the other users. The present system further provides multi-syncing for synchronization of a plurality of user accounts for sharing biorhythm data of each other for accurate and efficient communication. The present system controls (increase or decrease) an involuntary or unconscious physiological process by self-regulating and exercising control over physiological variables. The present invention provides a social platform to the users where they share their emotional data and allow other users to visualize the same to improve and work on their emotional state. Additionally, the present system improves communication amongst the users based on the biorhythmic data.

While embodiments of the present invention have been illustrated and described, it will be clear that the invention is not limited to these embodiments only. Numerous modifications, changes, variations, substitutions, and equivalents will be apparent to those skilled in the art, without departing from the scope of the invention, as described in the claims.

1. A system for collecting, analyzing and sharing biorhythm data among a plurality of users over a communication network, the system comprising:
a wearable user device to collect biorhythm data of the user; and
a computing device is communicatively connected with the wearable user device to receive the biorhythm data of the users over the communication network, wherein the computing device comprising:
a processor; and
a memory communicatively coupled to the processor, wherein the memory stores instructions executed by the processor, wherein the memory comprising:
a synchronization module to facilitate the users to access the biorhythm data, wherein the synchronization module comprising:
a storage module to store the biorhythm data of the users collected by the wearable user device corresponding to the plurality of users;
a categorization module to categorize the biorhythm data stored in the storage module into a plurality of profiles associated with each user;
a computation module to compute the biorhythm data; and
a communication module to communicate the computed data to a network platform, wherein the network platform facilitates the users to access the computed data and the profiles of the users connected to the network platform;
an artificial intelligence (AI) based agent module to establish an interaction with the users over the communication network, wherein the AI-based agent module comprising:
a tracking module to receive the biorhythm data from the wearable user device and monitor the interactions of a plurality of users and retrieves relevant data for analysis, wherein the tracking module is integrated with one or more messaging platforms and one or more voice platforms of the computing device corresponding to the users to monitor textual interactions and audio interactions of the users, wherein the tracking module processes the relevant data and the retrieved parameters to generate training data;
a software learning agent module to receive and process the training data to determine the emotional state of the user in a plurality of scenarios;
a virtual chat-bot module to initiate the interaction with the user and assist the user based on the learned data received from the software learning agent module; and
a community module to facilitate the user to connect and interact with a plurality of other users, wherein the community module facilitates the plurality of users to interact with each other and share emotional state and biorhythm data among the other users over the communication network;
an emotional data displaying module to analyze and display emotional data of the users in real-time, wherein the emotional data displaying module comprising:
an algorithmic module to analyze the biorhythm data and compute an emotional score of the user to generate one or more insights, wherein the emotional score is indicative of the emotional state of the user during the interactions; and
a visualization module to graphically represent a plurality of emotional cycles for a specific time-duration for the user, wherein the visualization module displays the insights and emotional scores of the users on the computing device associated with the users; and
a feedback module configured with the wearable user device to modulate biorhythms of the users based on the feedback emitted from the computing device, wherein the feedback module comprising:
a physiological data collection engine to collect physiological data of at least one physiological property of the user;
a biosignal generating engine to process the physiological data into at least one biosignal;
a feedback activation determining engine to monitor and measure the biosignal for a feedback activation condition; and
a feedback generating engine to trigger feedback upon satisfying a feedback activation condition, wherein the feedback activation condition triggers the feedback when the measured value is more than one or more predetermined threshold values.

2. The system according to claim 1, wherein the tracking module retrieves a plurality of parameters of the users from the biorhythm data and monitored data, wherein the plurality of parameters comprising location of the user, biorhythm data of the user, personal and social behavior of the user, and environment, month, day, and time of the interactions.

3. The system according to claim 1, wherein the plurality of scenarios comprising contexts, situations, and environments, wherein the software learning agent module is adaptable to continuously learn the contexts, situations, and environments based on the received training data and stores the learned data in a database.

4. The system according to claim 1, wherein the virtual chat-bot module interacts with the user to assist to improve the emotional state of the user.

5. The system according to claim 1, wherein the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols.

6. A method to collect, analyze and share biorhythm data among a plurality of users over a communication network, the method comprising steps of:
collecting biorhythm data of the user through a wearable user device;
receiving the biorhythm data of the users through a computing device communicatively connected with the wearable user device over the communication network;
facilitating the users to access the biorhythm data through a synchronization module, wherein the synchronization module performs a plurality of steps comprising:
storing the biorhythm data of the users collected by the wearable user device corresponding to the plurality of users through a storage module;
categorizing the biorhythm data stored in the storage module into a plurality of profiles associated with each user through a categorization module;
computing the biorhythm data through a computation module; and
communicating the computed data to a network platform through a communication module, wherein the network platform facilitates the users to access the computed data and the profiles of the users connected to the network platform;
establishing an interaction with the users over the communication network through an artificial intelligence (AI) based agent module, wherein the AI-based agent module performs a plurality of steps comprising:
receiving the biorhythm data from the wearable user device and monitor the interactions of a plurality of users and retrieves relevant data for analysis through a tracking module, wherein the tracking module is integrated with one or more messaging platforms and one or more voice platforms of the computing device corresponding to the users to monitor textual interactions and audio interactions of the users, wherein the tracking module processes the relevant data and the retrieved parameters to generate training data;
receiving and processing the training data to determine the emotional state of the user in a plurality of scenarios through a software learning agent module;
initiating the interaction with the user and assist the user based on the learned data received from the software learning agent module through a virtual chat-bot module; and
facilitating the user to connect and interact with a plurality of other users through a community module, wherein the community module facilitates the plurality of users to interact with each other and share emotional state and biorhythm data among the other users over the communication network;
analyzing and displaying emotional data of the users in real-time through an emotional data displaying module, wherein the emotional data displaying module performs a plurality of steps comprising:
analyzing the biorhythm data and computing an emotional score of the user to generate one or more insights through an algorithmic module, wherein the emotional score is indicative of the emotional state of the user during the interactions; and
graphically representing a plurality of emotional cycles for a specific time-duration for the user through a visualization module, wherein the visualization module displays the insights and emotional scores of the users on the computing device associated with the users; and
modulating biorhythms of the users based on the feedback emitted from the computing device through a feedback module, wherein the feedback module performs a plurality of steps comprising:
collecting physiological data of at least one physiological property of the user through a physiological data collection engine;
processing the physiological data into at least one biosignal through a biosignal generating engine;
monitoring and measuring the biosignal for a feedback activation condition through a feedback activation determining engine; and
triggering feedback upon satisfying a feedback activation condition through a feedback generating engine, wherein the feedback activation condition triggers the feedback when the measured value is more than one or more predetermined threshold values.

7. The method according to claim 6, wherein the tracking module retrieves a plurality of parameters of the users from the biorhythm data and monitored data, wherein the plurality of parameters comprising location of the user, biorhythm data of the user, personal and social behavior of the user, and environment, month, day, and time of the interactions.

8. The method according to claim 6, wherein the plurality of scenarios comprising contexts, situations, and environments, wherein the software learning agent module is adaptable to continuously learn the contexts, situations, and environments based on the received training data and stores the learned data in a database.

9. The method according to claim 6, wherein the virtual chat-bot module interacts with the user to assist to improve the emotional state of the user.

10. The method according to claim 6, wherein the visualization module displays emotional data in a plurality of manners on at least one of a two-dimensional (2D) graph, and a three-dimensional (3D) graphs by using at least one of a plurality of alpha-numeric characters, a plurality of geometric shapes, a plurality of holograms, and a plurality of symbols.
